(19) United States
(12) Patent Application Publication
(10) Pub. No.: US 2026/0048227 A1
(43) Pub. Date: Feb. 19, 2026

(54) HUMIDIFIER WITH CHEMICAL & ADHESIVE FREE TUBE CONNECTOR FOR MEDICAL EQUIPMENT

(71) Applicants: VINCENT MEDICAL (DONG GUAN) TECHNOLOGY CO., LTD., Dongguan, Guangdong (CN); VINCENT MEDICAL ADMIRED CO., LTD., Dongguan, Guangdong (CN); VINCENT MEDICAL (DONG GUAN) MANUFACTURING CO., LTD., Dongguan, Guangdong (CN)

(72) Inventors: Kam Man NG, Dongguan, Guangdong (CN); Zhiwei FENG, Dongguan, Guangdong (CN); CHEUNG TAI RAYMOND CHOI, Dongguan, Guangdong (CN); SIU TUNG LI, Dongguan, Guangdong (CN)

(21) Appl. No.: 18/996,617

(22) PCT Filed: Aug. 22, 2024
(86) PCT No.: PCT/CN2024/114039

§ 371 Date: Jan. 17, 2025

(30) Foreign Application Priority Data
Aug. 16, 2024 (CN) 202411130829.3

(51) Int. Cl. A61M 16/16; A61M 16/08

(52) U.S. Cl. CPC A61M 16/16; A61M 16/0816; A61M 16/0875; A61M 2202/0208

(57) ABSTRACT

A humidifier with a glue-free chemical solvent-free dropper bottle tube connector and respiratory equipment are provided, which relate to the technical field of respiratory equipment. The humidifier includes an infusion tube; a humidifier body, having an upper cover end and a base plate end arranged opposite to each other; a water inlet, fixedly provided on the upper cover end, where the water inlet is provided with a water inlet hole, and a plurality of locking parts are fixedly arranged on the water inlet; an infusion tube connector, having an insertion section, a limiting boss, and a hole entrance section arranged in sequence from top to bottom, where the hole entrance section is detachably connected to the water inlet hole, and the insertion section is detachably connected to the infusion tube; and a connector locking fastener.

### TECHNICAL FIELD

The present disclosure relates to the technical field of respiratory equipment, and in particular, to a humidifier with a glue-free chemical solvent-free dropper bottle tube connector and respiratory equipment.

### BACKGROUND

At present, an assembly process for a humidifier and an infusion tube generally adopts glue for curing and connecting. To ensure stable curing and connecting of the glue, the assembly process for the humidifier and the infusion tube usually needs to be air-dried for a long time or irradiated by using UV light to accelerate curing.

However, after assembly is completed, an existing glue curing process has a problem of volatilization of volatile organic matters, which poses a safety risk for a patient when using the humidifier.

Therefore, a conventional art has defects and disadvantages, which needs to be further improved and developed.

### SUMMARY

In view of the foregoing disadvantages in a conventional art, an objective of the present disclosure is to provide a glue-free chemical solvent-free dropper bottle tube connector and respiratory equipment, which aims to solve a problem of volatilization of volatile organic matters in an existing assembly process for a humidifier and an infusion tube in the conventional art.

A technical solution adopted in the present disclosure for solving the technical problem is as follows: a humidifier with a glue-free chemical solvent-free dropper bottle tube connector, configured for respiratory equipment. The humidifier includes:
an infusion tube;
a humidifier body, where the humidifier body has an upper cover end and a base plate end arranged opposite to each other;
a water inlet, where the water inlet is fixedly provided on the upper cover end, the water inlet is provided with a water inlet hole, and a plurality of locking parts are fixedly arranged on the water inlet;
an infusion tube connector, where the infusion tube connector has an insertion section, a limiting boss, and a hole entrance section arranged in sequence from top to bottom, the hole entrance section is detachably connected to the water inlet hole, and the insertion section is detachably connected to the infusion tube; and
a connector locking fastener, where the connector locking fastener is provided with a perforation hole, a plurality of fixing pieces are fixedly arranged on the connector locking fastener, and the plurality of fixing pieces are configured to cooperate with the locking parts and the infusion tube connector to fix the infusion tube to the water inlet hole.

Optionally, the infusion tube connector includes an ultrasonic line, where the ultrasonic line is annularly arranged at an end of the limiting boss deviating from the insertion section, and the ultrasonic line is configured to perform ultrasonic welding on the water inlet; and
an inverted fastener, where the inverted fastener is fixedly arranged on the insertion section.

Optionally, a cross section of the ultrasonic line along an axial direction of the infusion tube connector is arranged in a V shape, a triangle, or a cone shape.

Optionally, a diameter of the ultrasonic line is set as 1.2 times-2 times that of the water inlet hole.

Optionally, each of the plurality of locking parts includes a locking boss, and the locking boss is fixedly provided on a side wall of the water inlet.

Optionally, each of the plurality of fixing pieces includes a plurality of rotary fasteners, the plurality of rotary fasteners are fixedly arranged on an inner side wall of the connector locking fastener, and the plurality of rotary fasteners are configured to cooperate with the locking bosses and the infusion tube connector to fix the infusion tube to the water inlet hole.

The infusion tube penetrates out from the perforation hole, the infusion tube is sleeved in the insertion section, the hole entrance section is inserted into the water inlet hole, the limiting boss is propped against a hole edge of the water inlet hole, ultrasonic welding is performed on the water inlet and the infusion tube connector by using the ultrasonic line, the connector locking fastener is sleeved over the water inlet, and the plurality of rotary fasteners are rotated into the locking bosses by rotating the connector locking fastener.

Optionally, each of the plurality of locking parts includes a clamping boss, and the clamping boss is provided on a side wall of the water inlet.

Optionally, each of the plurality of fixing pieces includes a clamping groove, the plurality of clamping grooves are provided in a side wall of the connector locking fastener, the clamping bosses are adapted to the clamping grooves in dimensions, and the plurality of clamping grooves are configured to cooperate with the clamping bosses and the infusion tube connector to fix the infusion tube to the water inlet hole.

The infusion tube penetrates out from the perforation hole, the infusion tube is sleeved in the insertion section, the hole entrance section is inserted into the water inlet hole, the limiting boss is propped against a hole edge of the water inlet hole, ultrasonic welding is performed on the water inlet and the infusion tube connector by using the ultrasonic line, the connector locking fastener is sleeved over the water inlet, and the plurality of clamping grooves are all buckled in the clamping bosses.

Optionally, each of the plurality of locking parts includes a screw threads, and the screw threads are provided on a side wall of the water inlet.

Each of the plurality of fixing pieces includes a threaded groove, the plurality of threaded grooves are provided on an inner side wall of the connector locking fastener, the threaded grooves are adapted to the thread screws in dimensions, and the plurality of threaded grooves are configured to cooperate with the thread screws and the infusion tube connector to fix the infusion tube to the water inlet hole.

The infusion tube penetrates out from the perforation hole, the infusion tube is sleeved in the insertion section, the hole entrance section is inserted into the water inlet hole, the limiting boss is propped against a hole edge of the water inlet hole, ultrasonic welding is performed on the water inlet and the infusion tube connector by using the ultrasonic line, the connector locking fastener is sleeved over the water inlet, and the plurality of threaded grooves are all screwed into the screw threads.

Another technical solution adopted in the present disclosure for solving the technical problem is as follows: respiratory equipment includes the foregoing humidifier with a glue-free chemical solvent-free dropper bottle tube connector.

Compared with the conventional art, the present disclosure provides a humidifier with a glue-free chemical solvent-free dropper bottle tube connector and respiratory equipment. A stable connection between the humidifier and an infusion tube is achieved through cooperation of an infusion tube connector, a connector locking fastener, and a locking part, which avoids a problem of loosening that may be caused by a traditional connection using glue, a curing process using the glue is abandoned, and a novel humidifier with a glue-free chemical solvent-free dropper bottle tube connector is provided, so that a safety risk caused by volatilization of volatile organic matters is eliminated, safety of a user is ensured, the use of a glue curing process is avoided, a problem caused by the volatilization of the volatile organic matters is eliminated, equipment safety is improved, air-drying curing time is not required, manufacturing time and cost for the humidifier are reduced, and production capacity of products of the humidifier is improved.

### BRIEF DESCRIPTION OF THE DRAWINGS

FIG. 1 is an exploded view of a three-dimensional structure of a humidifier with a glue-free chemical solvent-free dropper bottle tube connector according to the present disclosure.

FIG. 2 is another exploded view of a three-dimensional structure of a humidifier with a glue-free chemical solvent-free dropper bottle tube connector according to the present disclosure.

FIG. 3 is a schematic diagram of a three-dimensional structure of an infusion tube connector of a humidifier with a glue-free chemical solvent-free dropper bottle tube connector according to the present disclosure.

FIG. 4 is a front view of a humidifier with a glue-free chemical solvent-free dropper bottle tube connector according to the present disclosure.

FIG. 5 is a cross-sectional view of FIG. 4 in I-I direction.

FIG. 6 is another exploded view of a three-dimensional structure of a humidifier with a glue-free chemical solvent-free dropper bottle tube connector according to the present disclosure.

FIG. 7 is another exploded view of a three-dimensional structure of a humidifier with a glue-free chemical solvent-free dropper bottle tube connector according to the present disclosure.

FIG. 8 is another exploded view of a three-dimensional structure of a humidifier with a glue-free chemical solvent-free dropper bottle tube connector according to the present disclosure.

Reference signs in the drawings:
10, humidifier; 11, infusion tube; 12, humidifier body; 121, upper cover end ; 122, base plate end; 13, water inlet; 131, locking part; 1311, locking boss; 1312, clamping boss; 1313, screw thread; 132, water inlet hole; 14, infusion tube connector; 141, insertion section; 142, limiting boss; 1421, ultrasonic line; 1422, inverted fastener; 143, hole entrance section; 15, connector locking fastener; 151, perforation hole; 152, fixing piece; 1521, rotary fastener; 1522, clamping groove; and 1523, threaded groove.

### DETAILED DESCRIPTION OF THE EMBODIMENTS

Embodiments of the present disclosure are described in detail below, and examples of the embodiments are shown in accompanying drawings, where the same or similar reference numerals throughout the description represent the same or similar elements or the elements having the same or similar functions. The embodiments described below with reference to the accompanying drawings are exemplary, and are only used for explaining the present disclosure and cannot be construed as a limitation to the present disclosure.

In descriptions of the present disclosure, it is to be understood that orientations or positional relationships indicated by terms “center”, “longitudinal”, “transverse”, “upper”, “lower”, “front”, “rear”, “left”, “right”, “vertical”, “horizontal”, “top”, “bottom”, “inner”, “outer”, and the like are the orientations or positional relationships shown based on the accompanying drawings, and are merely for the convenience of describing the present disclosure and simplifying the descriptions, rather than indicating or implying that the devices or elements referred to need to have particular orientations, and constructed and operated in particular orientations. Therefore, it cannot be construed as a limitation to the present disclosure. In addition, terms “first” and “second” are used merely for the purpose of description, and cannot be construed as indicating or implying relative importance or implying a quantity of indicated technical features. Therefore, a feature limited by “first” or “second” may explicitly or implicitly include one or more of the features. In the descriptions of the present disclosure, unless otherwise specified, “a plurality of” means two or more.

In the descriptions of the present disclosure, it is to be noted that, unless otherwise specified and limited, terms “mount”, “interconnect”, and “connect” are to be broadly understood. For example, the terms may refer to fixed connection and may also refer to detachable connection or integration; the terms may refer to mechanical connection and may alternatively refer to electrical connection; the terms may refer to direct mutual connection, may alternatively refer to indirect connection through a medium, and may refer to communication in two components. For those of ordinary skill in the art, specific meanings of the foregoing terms in the present disclosure may be understood according to specific situations.

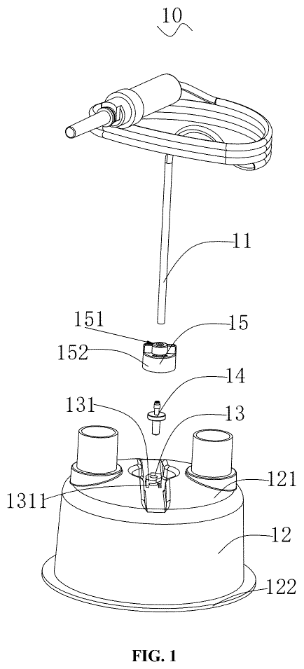

Referring to FIG. 1 to FIG. 3, a first embodiment of the present disclosure provides a humidifier 10 with a glue-free chemical solvent-free dropper bottle tube connector, configured for respiratory equipment. The humidifier 10 includes a humidifier body 12, a water inlet 13, an infusion tube connector 14, and a connector locking fastener 15. The humidifier body 12 has an upper cover end 121 and a base plate end 122. The water inlet 13 is fixed to the upper cover end 121. The water inlet 13 is provided with a water inlet hole 132 and a plurality of locking parts 131. The plurality of locking parts 131 are configured to connect an infusion tube 11 and the humidifier 10. The infusion tube connector 14 includes an insertion section 141, a limiting boss 142, and a hole entrance section 143. The infusion tube 11 is inserted into the infusion tube connector 14 through the insertion section 141, and the infusion tube 11 is prevented from being inserted too deeply by using the limiting boss 142. Next, the hole entrance section 143 of the infusion tube connector 14 is inserted into the water inlet hole 132 of the humidifier 10. The limiting boss 142 leans against a hole edge of the water inlet hole 132 to provide additional stability. To ensure connection stability, the locking part 131 on the side wall of the water inlet 13 cooperates with a connector locking fastener 15, and the connector locking fastener 15 is sleeved over the water inlet 13. Fixing pieces 152 on the locking fastener cooperate with the locking parts 131 by rotating or in another fixing manner, so as to firmly fix the infusion tube connector 14 to the water inlet hole 132. Meanwhile, due to a design of a perforation hole 151 in the connector locking fastener 15, the infusion tube 11 can penetrate out from this hole, so as to ensure positioning and fixing of the infusion tube 11. In this mechanical connection manner, a novel humidifier is provided, so that the use of a glue curing process is avoided, a problem about volatile organic matters is eliminated, equipment safety is improved, air-drying curing time is not required, manufacturing time and cost for the humidifier 10 are reduced, and production capacity of products of the humidifier 10 is improved.

Referring to FIG. 1 to FIG. 4, the humidifier 10 with a glue-free chemical solvent-free dropper bottle tube connector includes an infusion tube 11, a humidifier body 12, a water inlet 13, an infusion tube connector 14, and a connector locking fastener 15. The infusion tube 11 is configured to delivery liquid into the humidifier body 12. Specifically, the liquid may be distilled water, sterile water, distilled water mixed with liquid medicine, liquid medicine, or the like. The humidifier body 12 is configured to humidify gas. The humidifier body 12 has an upper cover end 121 and a base plate end 122 arranged opposite to each other. The base plate end 122 is configured to mount a heat conducting base plate. The water inlet 13 is configured to infuse the liquid guided by the infusion tube 11 into the humidifier body 12. The water inlet 13 is fixedly provided on the upper cover end 121. The water inlet 13 is provided with a water inlet hole 132. A plurality of locking parts 131 are fixedly arranged on the water inlet 13. The infusion tube connector 14 is configured to connect the infusion tube 11 to the water inlet 13. The infusion tube connector 14 has an insertion section 141, a limiting boss 142, and a hole entrance section 143. The insertion section 141, the limiting boss 142, and the hole entrance section 143 are arranged in sequence from top to bottom. The hole entrance section 143 is detachably connected to the water inlet hole 132, and the insertion section 141 is detachably connected to the infusion tube 11, so that the infusion tube 11 and the connector locking fastener 15 are easy to detach and maintain, service life of the humidifier 10 is prolonged, and convenience of the humidifier 10 is improved. The connector locking fastener 15 is provided with a perforation hole 151, and the perforation hole 151 is configured for the infusion tube 11 to penetrate out. A plurality of fixing pieces 152 are fixedly arranged on the connector locking fastener 15. The plurality of fixing pieces 152 are configured to cooperate with the locking parts 131 and the infusion tube connector 14 to fix the infusion tube 11 to the water inlet hole 132, and then a stable connection between the humidifier 10 and the infusion tube 11 is achieved through the cooperation of the infusion tube connector 14, the connector locking fastener 15, and the locking part 131, a problem of loosening that may be caused by a traditional glue connection is avoided, a curing process using glue is abandoned, a safety risk caused by volatilization of volatile organic matters is eliminated, user safety is ensured, air-drying curing time is not required, manufacturing time and cost for the humidifier 10 are reduced, and production capacity of products of the humidifier 10 is improved.

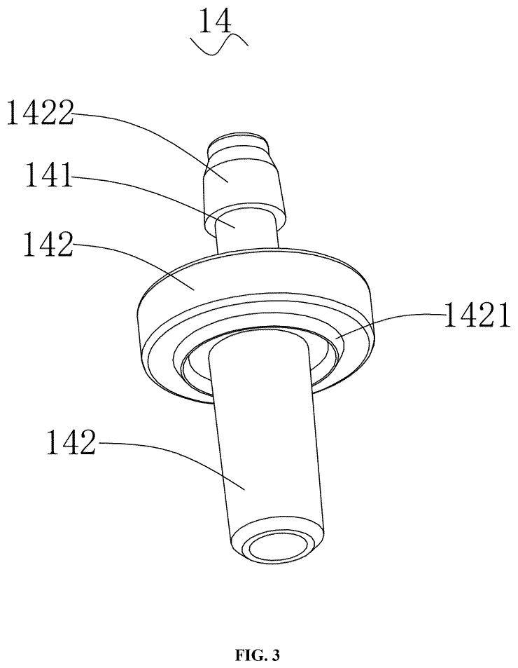

Referring to FIG. 3, in some embodiments, the infusion tube connector 14 further includes an ultrasonic line 1421 and an inverted fastener 1422. The ultrasonic line 1421 is arranged at an end of the limiting boss 142 deviating from the insertion section 141. The ultrasonic line 1421 is arranged annularly. The ultrasonic line 1421 is configured for ultrasonic welding with the water inlet 13. The inverted fastener 1422 is configured to prevent the infusion tube 11 from separating from the infusion tube connector 14. The inverted fastener 1422 is fixedly arranged on the insertion section 141, so that a curing process using glue is abandoned, a safety risk caused by volatilization of volatile organic matters is eliminated, user safety is ensured, air-drying curing time is not required, manufacturing time and cost for the humidifier 10 are reduced, and production capacity of products of the humidifier 10 is improved.

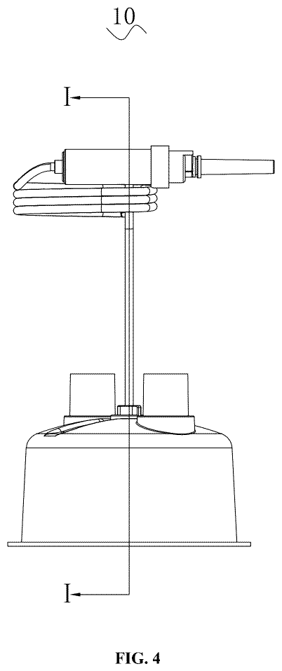

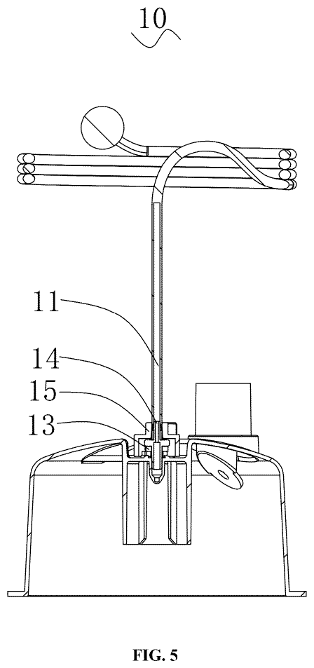

Referring to FIG. 4 and FIG. 5, in some embodiments, the inverted fastener 1422 is arranged in a cone shape. A maximum diameter of the inverted fastener 1422 is slightly greater than that of the infusion tube 11, thereby effectively preventing the infusion tube 11 from being unplugged from the inverted fastener 1422 after being stressed, and improving assembly efficiency between the infusion tube 11 and the infusion tube connector 14.

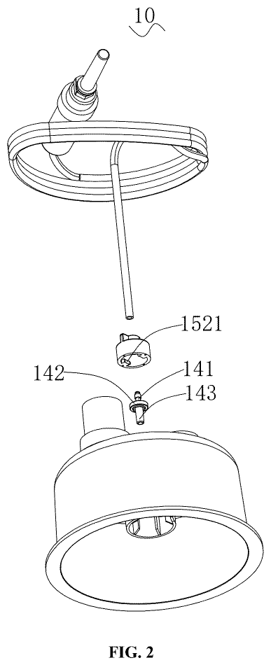

Referring to FIG. 2, in some embodiments, a cross section of the ultrasonic line 1421 along an axial direction of the infusion tube connector 14 is arranged in a V shape, a triangle, or a cone shape, which helps to concentrate and transfer energy during ultrasonic welding, and improves welding efficiency and quality, so that a connection between the infusion tube connector 14 and the water inlet 13 is more stable, a curing process using glue is abandoned, a safety risk caused by volatilization of volatile organic matters is eliminated, user safety is ensured, air-drying curing time is not required, manufacturing time and cost for the humidifier 10 are reduced, and production capacity of products of the humidifier 10 is improved.

Referring to FIG. 2, in some embodiments, the sharper the tip of the ultrasonic line 1421, the better, so that vibration energy may be concentrated at the tip of a triangle, then a uniform plastic melt flow is formed on the overall welding interface by accumulated heat, welding efficiency and quality are improved, a connection between the infusion tube connector 14 and the water inlet 13 is more stable, a curing process using glue is abandoned, a safety risk caused by volatilization of volatile organic matters is eliminated, user safety is ensured, air-drying curing time is not required, manufacturing time and cost for the humidifier 10 are reduced, and production capacity of products of the humidifier 10 is improved.

Referring to FIG. 2, in some embodiments, a welding principle of the ultrasonic line 1421 is that high-frequency friction is performed between the infusion tube connector 14 and the water inlet 13 to convert mechanical energy into thermal energy by using high-frequency vibration of a welding head of the ultrasonic line 1421, the thermal energy dissolves molecules on a welding surface between the infusion tube connector 14 and the water inlet 13 to recover activity of the molecules, and then the molecules are entangled with each other to achieve a welding purpose under assistance of an external acting force, so that air tightness and welding quality between the infusion tube connector 14 and the water inlet 13 are greatly improved, a problem of loosening that may be caused by a traditional glue connection is avoided, a curing process using glue is abandoned, a safety risk caused by volatilization of volatile organic matters is eliminated, user safety is ensured, equipment safety is improved, air-drying curing time is not required, manufacturing time and cost for the humidifier 10 are reduced, and production capacity of products of the humidifier 10 is improved.

Referring to FIG. 2, in some embodiments, a diameter of the ultrasonic line 1421 is set as 1.2 times-2 times that of the water inlet hole 132, that is, the ultrasonic line 1421 is arranged on an outer side of the water inlet hole 132, thereby optimizing a welding effect between the infusion tube connector 14 and the water inlet 13, ensuring strength and air tightness of a connection position between the infusion tube connector 14 and the water inlet 13, and improving reliability of an overall structure. The diameter of the ultrasonic line 1421 is greater than that of the water inlet hole 312, which can uniformly transfer stress during welding, stress concentration phenomena are reduced, and anti-fatigue performance of the connection position between the infusion tube connector 14 and the water inlet 13 is improved.

Referring to FIG. 1 to FIG. 5, in some embodiments, each of the plurality of locking parts 131 includes a locking boss 1311, and the locking boss 1311 is fixedly provided on a side wall of the water inlet 13, so that the infusion tube connector 14 can be quickly mounted and fixed, an assembly process is simplified, and production efficiency is improved.

Referring to FIG. 1 to FIG. 5, in some embodiments, each of the plurality of fixing pieces 152 includes a plurality of rotary fasteners 1521. The plurality of rotary fasteners 1521 are fixedly arranged on an inner side wall of the connector locking fastener 15, and the plurality of rotary fasteners 1521 are configured to cooperate with the locking boss 1311 and the infusion tube connector 14 to fix the infusion tube 11 to the water inlet hole 132. The infusion tube 11 penetrates out from the perforation hole 151, the infusion tube 11 is sleeved in the insertion section 141, the hole entrance section 143 is inserted into the water inlet hole 132, the limiting boss 142 is propped against a hole edge of the water inlet hole 132, ultrasonic welding is performed on the water inlet 13 and the infusion tube connector 14 by using the ultrasonic line 1421, the connector locking fastener 15 is sleeved over the water inlet 13, and the plurality of rotary fasteners 1521 are rotated into the locking bosses 1311 by rotating the connector locking fastener 15. Through the cooperation of the infusion tube connector 14, the locking bosses 1311, and the rotary fasteners 1521, a stable connection between the humidifier 10 and the infusion tube 11 is achieved, a problem of loosening that may be caused by a traditional glue connection is avoided, a curing process using glue is abandoned, a safety risk caused by volatilization of volatile organic matters is eliminated, user safety is ensured, equipment safety is improved, air-drying curing time is not required, manufacturing time and cost for the humidifier 10 are reduced, and production capacity of products of the humidifier 10 is improved.

In some embodiments, the locking boss 1311 includes a horizontal boss, a vertical boss, and a clamping boss. The horizontal boss and the vertical boss are provided perpendicularly to form a locking space. The horizontal boss and the vertical boss are integrally arranged. The limiting boss 142 is arranged on the upper cover end 121. The limiting boss 142 is configured to prevent the plurality of rotary fasteners 1521 from sliding out from the locking space, thereby effectively preventing the infusion tube 11 from falling during use, and improving safety and reliability of a connection between the infusion tube connector 14 and the water inlet 13.

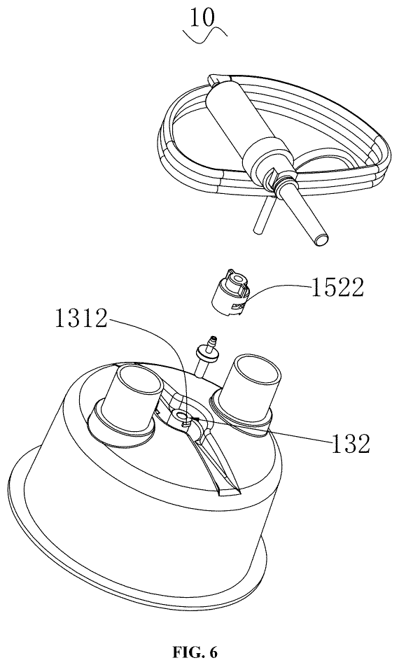

Referring to FIG. 6, in some embodiments, each of the plurality of locking parts 131 includes a clamping boss 1312, and the clamping boss 1312 is provided on a side wall of the water inlet 13, so that the infusion tube connector 14 can be quickly mounted and fixed, an assembly process is simplified, and production efficiency is improved.

Referring to FIG. 6, in some embodiments, an end face of the clamping boss 1312 is flush with an end face of the water inlet 13, so that a clamping difficulty between the connector locking fastener 15 and the water inlet 13 is reduced, the infusion tube connector 14 can be quickly mounted and fixed, an assembly process is simplified, and production efficiency is improved.

Referring to FIG. 6, in some embodiments, each of the plurality of fixing pieces 152 includes a clamping groove 1522. The plurality of clamping grooves 1522 are provided in a side wall of the connector locking fastener 15. The clamping bosses 1312 are adapted to the clamping grooves 1522 in dimensions. The plurality of clamping grooves 1522 are configured to cooperate with the clamping boss 1312 and the infusion tube connector 14 to fix the infusion tube 11 to the water inlet hole 132. The infusion tube 11 penetrates out from the perforation hole 151. The infusion tube 11 is sleeved in the insertion section 141. The hole entrance section 143 is inserted into the water inlet hole 132. The limiting boss 142 is propped against a hole edge of the water inlet hole 132. Ultrasonic welding is performed on the water inlet 13 and the infusion tube connector 14 by using the ultrasonic line 1421. The connector locking fastener 15 is sleeved over the water inlet 13. The plurality of clamping grooves 1522 are all buckled in the clamping bosses 1312, so that air tightness and welding quality between the infusion tube connector 14 and the water inlet 13 are greatly improved, a problem of loosening that may be caused by a traditional glue connection is avoided, a curing process using glue is abandoned, a safety risk caused by volatilization of volatile organic matters is eliminated, user safety is ensured, safety of the humidifier 10 is improved, air-drying curing time is not required, manufacturing time and cost for the humidifier 10 are reduced, and production capacity of products of the humidifier 10 is improved.

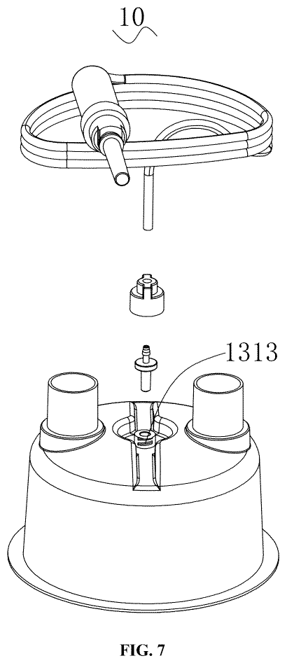

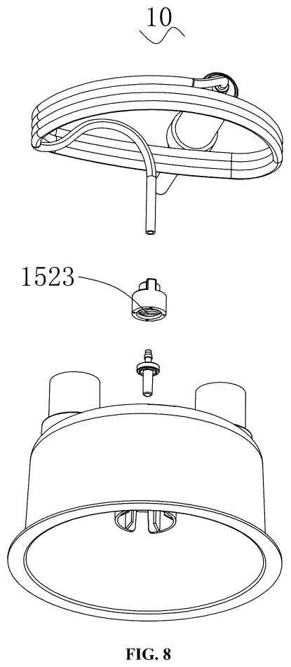

Referring to FIG. 7 and FIG. 8, in some embodiments, each of the plurality of locking parts 131 includes screw threads 1313. The screw threads 1313 are provided on a side wall of the water inlet 13. Each of the plurality of fixing pieces 152 includes a threaded groove 1523. The plurality of threaded grooves 1523 are provided on an inner side wall of the connector locking fastener 15. The threaded grooves 1523 are adapted to the screw threads 1313 in dimensions. The plurality of threaded grooves 1523 are configured to cooperate with the screw threads 1313 and the infusion tube connector 14 to fix the infusion tube 11 to the water inlet hole 132, so that air tightness between the water inlet 13 and the connector locking fastener 15 is improved, liquid leakage is prevented, and safety and reliability of the humidifier 10 are improved. The infusion tube 11 penetrates out from the perforation hole 151, the infusion tube 11 is sleeved in the insertion section 141, the hole entrance section 143 is inserted into the water inlet hole 132, the limiting boss 142 is propped against a hole edge of the water inlet hole 132, ultrasonic welding is performed on the water inlet 13 and the infusion tube connector 14 by using the ultrasonic line 1421, the connector locking fastener 15 is sleeved over the water inlet 13, and the plurality of threaded grooves 1523 are all screwed into the screw threads 1313, so that air tightness and welding quality between the infusion tube connector 14 and the water inlet 13 are greatly improved, a problem of loosening that may be caused by a traditional glue connection is avoided, a curing process using glue is abandoned, a safety risk caused by volatilization of volatile organic matters is eliminated, user safety is ensured, air-drying curing time is not required, manufacturing time and cost for the humidifier 10 are reduced, and production capacity of products of the humidifier 10 is improved.

In some embodiments, “glue-free chemical solvent-free” in the humidifier 10 with a glue-free chemical solvent-free dropper bottle tube connector in the present disclosure refers to that glue or other chemical solvents are not used during an assembly process of the humidifier body 12 and the infusion tube 11, so that a problem of loosening that may be caused by a traditional glue connection is avoided, a curing process using glue is abandoned, a safety risk caused by volatilization of volatile organic matters is eliminated, user safety is ensured, air-drying curing time is not required, manufacturing time and cost for the respiratory equipment are reduced, and production capacity of the respiratory equipment is improved.

A second embodiment of the present disclosure provides respiratory equipment, including the humidifier with a glue-free chemical solvent-free dropper bottle tube connector as described above, so that air tightness and welding quality between the infusion tube connector and the water inlet are greatly improved, a problem of loosening that may be caused by a traditional glue connection is avoided, a curing process using glue is abandoned, a safety risk caused by volatilization of volatile organic matters is eliminated, user safety is ensured, moreover, air-drying curing time is not required, manufacturing time and cost for the respiratory equipment are reduced, and production capacity of the respiratory equipment is improved.

In conclusion, the present disclosure provides a humidifier with a glue-free chemical solvent-free dropper bottle tube connector and respiratory equipment. The humidifier includes an infusion tube; a humidifier body, where the humidifier body has an upper cover end and a base plate end arranged opposite to each other; a water inlet, where the water inlet is fixedly provided on the upper cover end, the water inlet is provided with a water inlet hole, and a plurality of locking parts are fixedly arranged on the water inlet; an infusion tube connector, where the infusion tube connector has an insertion section, a limiting boss, and a hole entrance section arranged in sequence from top to bottom, the hole entrance section is detachably connected to the water inlet hole, and the insertion section is detachably connected to the infusion tube; and a connector locking fastener, where the connector locking fastener is provided with a perforation hole, a plurality of fixing pieces are fixedly arranged on the connector locking fastener, and the plurality of fixing pieces are configured to cooperate with the locking parts and the infusion tube connector to fix the infusion tube to the water inlet hole. A novel humidifier with a glue-free chemical solvent-free dropper bottle tube connector is provided, so that air tightness and welding quality between the infusion tube connector and the water inlet are greatly improved, a problem of loosening that may be caused by a traditional glue connection is avoided, a curing process using glue is abandoned, a safety risk caused by volatilization of volatile organic matters is eliminated, user safety is ensured, air-drying curing time is not required, manufacturing time and cost for the humidifier are reduced, and production capacity of products of the humidifier is improved.

It is to be understood that applications of the present disclosure are not limited to the foregoing examples. For those of ordinary skill in the art, improvements or transformations can be made according to the foregoing descriptions, and all these improvements and transformations fall within the scope of protection of the claims attached to the present disclosure.

1. A humidifier with a glue-free chemical solvent-free dropper bottle tube connector, configured for respiratory equipment, comprising:
an infusion tube;
a humidifier body, wherein the humidifier body has an upper cover end and a base plate end arranged opposite to each other;
a water inlet, wherein the water inlet is fixedly provided on the upper cover end, the water inlet is provided with a water inlet hole, and a plurality of locking parts are fixedly arranged on the water inlet;
an infusion tube connector, wherein the infusion tube connector has an insertion section, a limiting boss, and a hole entrance section arranged in sequence from top to bottom, the hole entrance section is detachably connected to the water inlet hole, and the insertion section is detachably connected to the infusion tube; and
a connector locking fastener, wherein the connector locking fastener is provided with a perforation hole, a plurality of fixing pieces are fixedly arranged on the connector locking fastener, and the plurality of fixing pieces are configured to cooperate with the locking parts and the infusion tube connector to fix the infusion tube to the water inlet hole.

2. The humidifier with a glue-free chemical solvent-free dropper bottle tube connector according to claim 1, wherein the infusion tube connector further comprises:
an ultrasonic line, wherein the ultrasonic line is annularly arranged at an end of the limiting boss deviating from the insertion section, and the ultrasonic line is configured for ultrasonic welding with the water inlet; and
an inverted fastener, wherein the inverted fastener is fixedly arranged on the insertion section.

3. The humidifier with a glue-free chemical solvent-free dropper bottle tube connector according to claim 2, wherein a cross section of the ultrasonic line along an axial direction of the infusion tube connector is arranged in a V shape, a triangle, or a cone shape.

4. The humidifier with a glue-free chemical solvent-free dropper bottle tube connector according to claim 2, wherein a diameter of the ultrasonic line is set as 1.2 times-2 times that of the water inlet hole.

5. The humidifier with a glue-free chemical solvent-free dropper bottle tube connector according to claim 2, wherein each of the plurality of locking parts comprises a locking boss, and the locking boss is fixedly provided on a side wall of the water inlet.

6. The humidifier with a glue-free chemical solvent-free dropper bottle tube connector according to claim 5, wherein
each of the plurality of fixing pieces comprises a plurality of rotary fasteners, the plurality of rotary fasteners are fixedly arranged on an inner side wall of the connector locking fastener, and the plurality of rotary fasteners are configured to cooperate with the locking boss and the infusion tube connector to fix the infusion tube to the water inlet hole, wherein
the infusion tube penetrates out from the perforation hole, the infusion tube is sleeved in the insertion section, the hole entrance section is inserted into the water inlet hole, the limiting boss is propped against a hole edge of the water inlet hole, ultrasonic welding is performed on the water inlet and the infusion tube connector by using the ultrasonic line, the connector locking fastener is sleeved over the water inlet, and the plurality of rotary fasteners are rotated into the locking bosses by rotating the connector locking fastener.

7. The humidifier with a glue-free chemical solvent-free dropper bottle tube connector according to claim 2, wherein each of the plurality of locking parts comprises a clamping boss, and the clamping boss is provided on a side wall of the water inlet.

8. The humidifier with a glue-free chemical solvent-free dropper bottle tube connector according to claim 7, wherein each of the plurality of fixing pieces comprises a clamping groove, the plurality of clamping grooves are provided in a side wall of the connector locking fastener, the clamping bosses are adapted to the clamping grooves in dimensions, and the plurality of clamping grooves are configured to cooperate with the clamping bosses and the infusion tube
the infusion tube penetrates out from the perforation hole, the infusion tube is sleeved in the insertion section, the hole entrance section is inserted into the water inlet hole, the limiting boss is propped against a hole edge of the water inlet hole, ultrasonic welding is performed on the water inlet and the infusion tube connector by using the ultrasonic line, the connector locking fastener is sleeved over the water inlet, and the plurality of clamping grooves are all buckled in the clamping bosses.

9. The humidifier with a glue-free chemical solvent-free dropper bottle tube connector according to claim 2, wherein each of the plurality of locking parts comprises screw threads, and the screw threads are provided on a side wall of the water inlet;
each of the plurality of fixing pieces comprises a threaded groove, the plurality of threaded grooves are provided on an inner side wall of the connector locking fastener, the threaded grooves are adapted to the screw threads in dimensions, and the plurality of threaded grooves are configured to cooperate with the screw threads and the infusion tube connector to fix the infusion tube to the water inlet hole, wherein
the infusion tube penetrates out from the perforation hole, the infusion tube is sleeved in the insertion section, the hole entrance section is inserted into the water inlet hole, the limiting boss is propped against a hole edge of the water inlet hole, ultrasonic welding is performed on the water inlet and the infusion tube connector by using the ultrasonic line, the connector locking fastener is sleeved over the water inlet, and the plurality of threaded grooves are all screwed into the screw threads.

10. Respiratory equipment, comprising the humidifier with a glue-free chemical solvent-free dropper bottle tube connector according to claim 1.
